(19) United States
(12) Patent Application Publication
(10) Pub. No.: US 2023/0143229 A1
(43) Pub. Date: May 11, 2023

(54) METHOD FOR DIAGNOSTIC ULTRASOUND OF CAROTID ARTERY

(71) Applicant: AIDOT INC., Seoul (KR)

(72) Inventor: Jae Hoon JEONG, Seongnam-si (KR)

(73) Assignee: AIDOT INC., Seoul (KR)

(21) Appl. No.: 17/912,577

(22) PCT Filed: Sep. 18, 2020
(86) PCT No.: PCT/KR2020/012613

§ 371 Date: Sep. 19, 2022

(30) Foreign Application Priority Data
Mar. 19, 2020 (KR) 10-2020-0033975

(51) Int. Cl. A61B 8/08; A61B 8/00; G16H 30/40; G06T 7/00; G06N 3/08; G06N 3/042

(52) U.S. Cl. CPC A61B 8/0891; A61B 8/461; A61B 8/5223; G16H 30/40; G06T 7/0012; G06N 3/08; G06N 3/042; G06T 2207/30101; G06T 2207/20084

(57) ABSTRACT

The present disclosure relates to a carotid artery diagnostic method, and more particularly to a method for diagnosing an abnormal symptom of a carotid artery using one or more artificial neural networks. A carotid ultrasound diagnostic method capable of being executed in a computer system capable of reading a carotid ultrasound video image includes extracting a carotid artery blood vessel image from an input or received carotid ultrasound video image or a carotid ultrasound video image accessed from a storage unit using a pre-trained artificial neural network, and diagnosing whether a carotid artery for the extracted carotid artery blood vessel image is normal using a pre-trained artificial neural network and displaying and outputting a diagnosis result.

### TECHNICAL FIELD

The present disclosure relates to a carotid artery diagnostic system, and more particularly to a method for diagnosing an abnormal symptom of a carotid artery using an artificial neural network.

### BACKGROUND ART

The carotid artery is an artery that passes through the neck and enters the face and skull, and is roughly divided into an external carotid artery and an internal carotid artery. The external carotid artery mainly supplies blood to the skin or muscles outside the skull, and the internal carotid artery supplies blood to the brain or nerve tissue within the skull.

Even if the external carotid artery is narrowed or blocked, there is no particular problem because blood is supplied relatively abundantly through other blood vessels. However, if the internal carotid artery is narrowed or blocked, blood supply to the brain may be reduced, and the adipose tissues deposited (stacked and adhered) on the wall of the internal carotid artery may fall off and flow to the distal end of the cerebral blood vessel to block the blood vessel. The narrowing of the carotid artery including the internal carotid artery as described above is referred to as a carotid artery stenosis, which reduces blood flow or blocks blood vessels to lead to ischemic stroke. Therefore, those with carotid artery stenosis are treated for the prevention and treatment of stroke.

Examples of a non-invasive method for diagnosing and examining the carotid artery stenosis include carotid ultrasound, brain computed tomography (CT), and brain magnetic resonance imaging (MRI), and examples of an invasive method include cerebral angiography.

Among the diagnosis and examination methods, the carotid ultrasound is used for an early diagnosis of cerebrovascular disease, and is a simple examination that observes the presence/absence of plaque in the carotid artery, blood flow, blood vessel thickness, etc. The carotid ultrasound has advantages of short examination time and low cost, but has disadvantage in that it can be performed only when an examiner acquires sufficient skills and knowledge. Further, the carotid ultrasound has disadvantages in that there may be a difference in reading ability between examiners even for the same ultrasound image, and in that even an experienced examiner has a high probability of being misdiagnosed due to poor reading ability for a sign minutely displayed on an ultrasound image.

Hence, there is needed a new type of carotid artery diagnostic system that can be conveniently used by anyone during a regular health checkup stage and can predict and diagnose abnormal symptoms of carotid artery by accurately detecting even the sign minutely displayed on the ultrasound image without being dependent on the reading ability of each examiner.

### PRIOR ART DOCUMENT

### Patent Document

(Patent Document 1) Korean Patent No. 10-1059824

(Patent Document 2) Korean Patent No. 10-2009840

### DISCLOSURE

### Technical Problem

An object of the present disclosure that is conceived in accordance with the above-described needs is to provide a carotid ultrasound diagnostic method using an artificial neural network capable of automatically diagnosing uniformly and accurately abnormal symptoms of a carotid artery irrespective of an examiner.

Another object of the present disclosure is to provide a carotid ultrasound diagnostic method using an artificial neural network that can detect a vascular plaque that is likely to develop into a floating thrombus and previously notify the possibility of a stroke.

Another object of the present disclosure is to provide a carotid ultrasound diagnostic method using an artificial neural network that can automatically diagnose whether a carotid artery is abnormal, and can display the abnormal carotid artery by differentiating a risk of the carotid artery in multiple levels.

Another object of the present disclosure is to provide a carotid ultrasound diagnostic method using an artificial neural network that can accurately and automatically diagnose whether there is an abnormality in a carotid artery using one or more artificial neural networks, or can accurately and automatically diagnose whether there is an abnormality in a carotid artery with respect to a carotid ultrasound video image transmitted from a remote location and notify a diagnosis result.

### Technical Solution

In order to achieve the above-described and other objects, a carotid ultrasound diagnostic method according to an embodiment of the present disclosure can be executed in a computer system capable of reading a carotid ultrasound video image, the carotid ultrasound diagnostic method comprising:

a first step of extracting a carotid artery blood vessel image from an input or received carotid ultrasound video image or a carotid ultrasound video image accessed from a storage unit using a pre-trained artificial neural network; and

a second step of diagnosing whether a carotid artery for the extracted carotid artery blood vessel image is normal using a pre-trained artificial neural network and displaying and outputting a diagnosis result.

In a modified embodiment, the carotid ultrasound diagnostic method further comprises:

a third step of, when the diagnosis result is abnormal, further diagnosing a carotid artery risk with respect to the extracted carotid artery blood vessel image using a pre-trained artificial neural network and displaying and outputting the diagnosed carotid artery risk as the diagnosis result.

In some cases, in the third step, a lesion area in the extracted carotid artery blood vessel image is displayed and output together.

The carotid ultrasound diagnostic method according to an embodiment of the present disclosure may further comprise, immediately before the third step, processing to expand a carotid artery blood vessel in the extracted carotid artery blood vessel image.

The carotid ultrasound diagnostic method according to an embodiment of the present disclosure may further comprise, before the second step, performing a heat-map processing on the extracted carotid artery blood vessel image.

In the carotid ultrasound diagnostic method described above, the first step uses a first artificial neural network that is trained by cropping an area set as a region of interest (ROI) by a medical specialist in one or more carotid ultrasound video images and setting a carotid artery blood vessel image that is denoised as learning data.

The second step uses a second artificial neural network that is trained by setting one or more carotid artery blood vessel images, that are marked as normal or abnormal by the medical specialist, as learning data.

The third step uses a third artificial neural network that is trained by setting a lesion area set by the medical specialist in one or more carotid artery blood vessel images and a carotid artery risk set for the lesion area, as learning data.

A carotid ultrasound diagnostic method according to another embodiment of the present disclosure can be executed in a computer system capable of reading a carotid ultrasound video image, the carotid ultrasound diagnostic method comprising:

diagnosing whether a carotid artery is normal in an input or received carotid ultrasound video image or a carotid ultrasound video image accessed from a storage unit using a pre-trained artificial neural network;

when a diagnosis result is abnormal, further diagnosing a carotid artery risk using the artificial neural network; and

displaying and outputting one of diagnosing whether the carotid artery is normal and diagnosing the carotid artery risk as the diagnosis result.

In the carotid ultrasound diagnostic method, a lesion area may be displayed and output together with the carotid artery risk.

When the diagnosis result is abnormal, the carotid artery risk may be diagnosed by processing to expand a carotid artery blood vessel capable of being extracted from the carotid ultrasound video image.

The carotid ultrasound diagnostic method, may further comprise, before diagnosing whether the carotid artery is normal, performing a heat-map processing on the carotid ultrasound video image.

### Advantageous Effects

According to the above-described means of solving the technical problems, a carotid ultrasound diagnostic method according to an embodiment of the present disclosure automatically diagnoses whether there is an abnormality in carotid artery with respect to a carotid ultrasound video image using one or more artificial neural networks and also displays a risk and a lesion area of the carotid artery together, and thus can provide an effect of accurately detecting even a sign minutely displayed on the carotid ultrasound video image without being dependent on the reading ability of each examiner (reader).

Furthermore, the present disclosure can be implemented by a method executable in a controller of an independent ultrasound diagnostic device and can also be implemented by a telemedicine server, and thus the present disclosure has an advantage of being able to provide telemedicine services. In addition, the present disclosure can detect and display in advance a thrombus with high separability that cannot be identified with the naked eye of an examiner, and thus the present disclosure can prevent in advance a stroke risk by taking necessary measures in advance for examinees with a possibility of thrombus floating, examinees with carotid artery stenosis, and the like.

### DESCRIPTION OF DRAWINGS

FIG. 1 is a block diagram illustrating configuration of a medical diagnostic device including a carotid ultrasound diagnostic system capable of implementing a method according to an embodiment of the present disclosure.

FIG. 2 illustrates configuration of a carotid ultrasound diagnostic system according to another embodiment of the present disclosure illustrated in FIG. 1.

FIG. 3 illustrates a learning process of a carotid artery extraction unit illustrated in FIG. 1.

FIG. 4 illustrates a learning process of a carotid artery diagnosis unit illustrated in FIG. 1.

FIG. 5 illustrates a learning process of a carotid artery diagnosis unit illustrated in FIG. 2.

FIG. 6 illustrates a diagnostic process of a carotid ultrasound diagnostic system according to an embodiment of the present disclosure.

FIGS. 7 to 13 illustrate an operation of a carotid ultrasound diagnostic system 200 according to an embodiment of the present disclosure.

### MODE FOR INVENTION

The following detailed description of the present disclosure refers to the accompanying drawings, which show by way of illustration a specific embodiment in which the present disclosure can be implemented, in order to clarify the objects, technical solutions and advantages of the present disclosure. These embodiments are described in detail to enable those skilled in the art to sufficiently implement the present disclosure.

And, ‘learning’ used throughout the detailed description and claims of the present disclosure is a term referring to performing deep learning according to a procedure, and thus a person skilled in the art will appreciate that the learning is not intended to refer to a mental action such as a human educational activity. Further, words of ‘include’ or ‘comprise’ and variations thereof used throughout the detailed description and claims of the present disclosure are not intended to exclude other technical features, additions, components or steps. A person skilled in the art will appreciate part of other objects, advantages and characteristics of the present disclosure through the present description and appreciate other part through embodiments of the present disclosure. The following illustrations and drawings are provided by way of illustration and are not intended to limit the present disclosure. Furthermore, the present disclosure encompasses all possible combinations of embodiments disclosed herein. It should be understood that various embodiments of the present disclosure are different from each other but do not need to be mutually exclusive. For example, specific structures and characteristics disclosed herein can be implemented in other embodiments without departing from the spirit and scope of the present disclosure with respect to one embodiment. It should be understood that the position or arrangement of individual components in respective embodiments disclosed herein can be changed without departing from the spirit and scope of the present disclosure. Accordingly, the following detailed description is not intended to be taken in a limiting sense, and the scope of the present disclosure, if properly described, is limited only by the appended claims, along with all scope equivalents to those claimed. Like reference numerals in the drawings refer to the same or similar functions throughout the various aspects.

In the present disclosure, unless otherwise indicated or clearly contradicted by context, items referred to in the singular encompass the plural, unless the context requires otherwise. It will be noted that a detailed description of known arts will be omitted if it is determined that the detailed description of the known arts can obscure embodiments of the present disclosure.

For reference, an artificial neural network mentioned below may be a convolutional neural network (CNN) model in which artificial neural networks are stacked in multiple layers. This can be expressed as a deep neural network in the sense of a network with a deep structure. In the deep neural network, features of each image are automatically learned by learning a large amount of data, and though this, the network is trained in a way of minimizing errors of an objective function. Since the CNN model is already known, a detailed description thereof will be omitted.

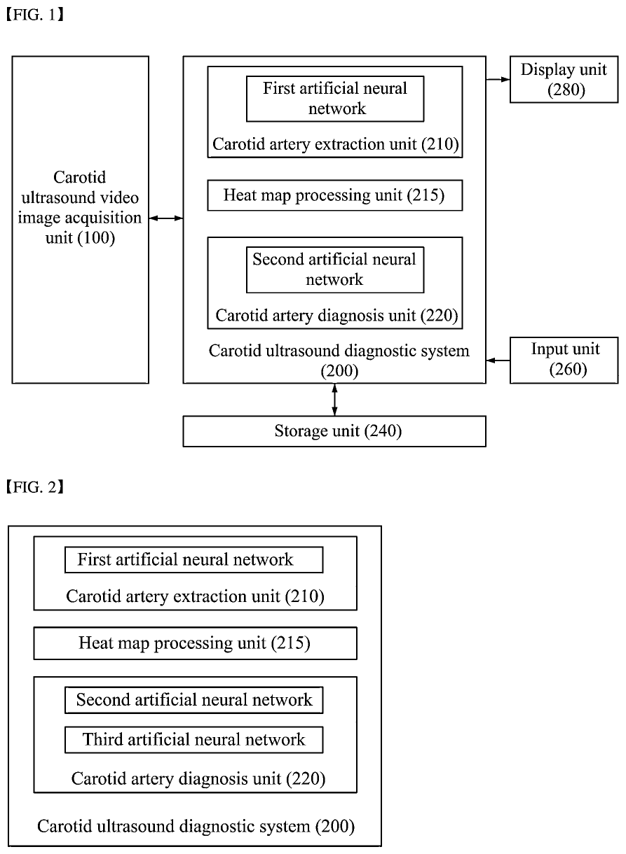

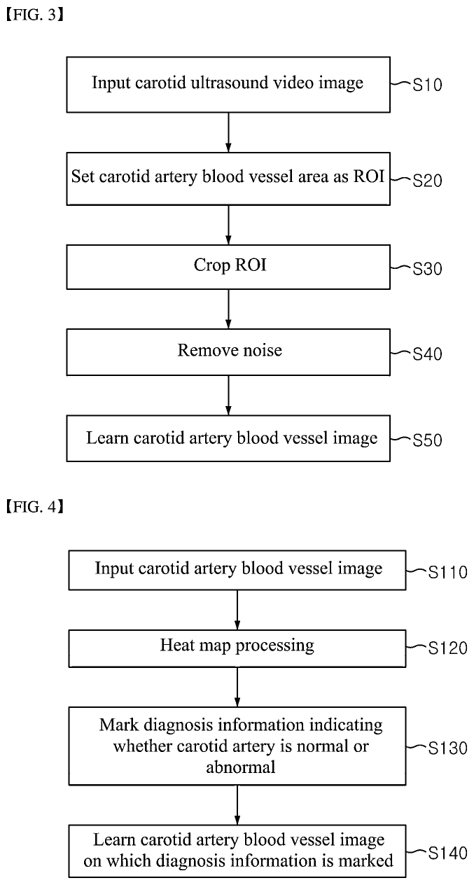

FIG. 1 is a block diagram illustrating configuration of a medical diagnostic device including a carotid ultrasound diagnostic system 200 capable of implementing a method according to an embodiment of the present disclosure. FIG. 2 illustrates configuration of a carotid ultrasound diagnostic system 200 according to another embodiment of the present disclosure illustrated in FIG. 1.

FIG. 1 illustrates a medical diagnostic device, for example, an ultrasound medical diagnostic device, in which a carotid ultrasound diagnostic method according to an embodiment of the present disclosure is implemented by the carotid ultrasound diagnostic system 200. However, the medical diagnostic device can be implemented in an executable way in a controller of a computer system that can diagnose symptoms by reading an input or received ultrasound video image or an ultrasound video image read from a memory, or can be implemented in an executable way in a remote diagnosis server that can be connected to computer systems of multiple medical institutions via a communication network for remote diagnosis, thereby diagnosing whether there is an abnormality in the carotid artery.

Referring to FIG. 1, a carotid ultrasound video image acquisition unit 100 is configured to acquire an ultrasound video image of the carotid artery to be diagnosed. If the carotid ultrasound diagnostic system 200 is a part of the medical diagnostic device, the carotid ultrasound video image acquisition unit 100 may be implemented by an ultrasound probe that transmits an ultrasound signal to an examination area including the carotid artery and receives an ultrasound echo signal reflected from the examination area, and an ultrasound video image generator that processes the ultrasound echo signal provided from the ultrasound probe and converts the processed ultrasound echo into an carotid ultrasound video image.

If the carotid ultrasound diagnostic system 200 is a computer system for diagnosis used by a medical specialist or a computer system of a medical institution, the carotid ultrasound video image acquisition unit 100 may be an interface unit that can data-interface with a peripheral ultrasound device including the ultrasound probe and the ultrasound video image generator, or an interface unit that can data-interface with a removable storage device.

If the carotid ultrasound diagnostic system 200 for implementing the carotid ultrasound diagnostic method is built on a remote diagnostic server, the carotid ultrasound video image acquisition unit 100 may be a receiving unit for receiving a carotid ultrasound video image from a computer system of a remote medical institution via a communication network.

Referring again to FIG. 1, the carotid ultrasound diagnostic system 200 includes:

a carotid artery extraction unit 210 configured to extract a carotid artery blood vessel image from the carotid ultrasound video image generated or transmitted or read from the carotid ultrasound video image acquisition unit 100 using a pre-trained first artificial neural network; and

a carotid artery diagnosis unit 220 configured to diagnose whether the carotid artery for the carotid artery blood vessel image is normal (normal or abnormal) using a pre-trained second artificial neural network and output a diagnosis result to a display unit 280.

The carotid artery extraction unit 210 including the first artificial neural network crops an area set as a region of interest (ROI) by the medical specialist in one or more carotid ultrasound video images and sets the carotid artery blood vessel image, that has been denoised, as learning data to train the first artificial neural network.

The carotid artery diagnosis unit 220 including the second artificial neural network sets one or more carotid artery blood vessel images, that are marked as normal or abnormal by the medical specialist, as learning data to train the second artificial neural network.

In a modified embodiment, as illustrated in FIG. 2, the carotid artery diagnosis unit 220 may further include a third artificial neural network in addition to the second artificial neural network. In this case, if the result of diagnosis using the second artificial neural network is abnormal, the carotid artery diagnosis unit 220 may diagnose a carotid artery risk with respect to the carotid artery blood vessel image using the pre-trained third artificial neural network and output the diagnosed risk to the display unit 280 as a diagnosis result.

The ‘carotid artery risk’ refers to a risk level indicated by grading the risk by stages, such as an ‘abnormal high-risk group’ and an ‘abnormal low-risk group’. In the following description, the carotid artery risk is graded into two levels. However, this is merely an example, and the carotid artery risk may be subdivided into two or more levels.

The carotid artery diagnosis unit 220 including the second and third artificial neural networks sets a lesion area set by the medical specialist in one or more carotid artery blood vessel images and the carotid artery risk set for the lesion area as learning data to train the third artificial neural network.

The carotid artery diagnosis unit 220 illustrated in FIG. 2 may display and output the lesion area (e.g., location of the plaque) in the carotid artery blood vessel image together with the carotid artery risk as the diagnosis result.

In addition, the carotid artery diagnosis unit 220 illustrated in FIG. 2 may process to expand a carotid artery blood vessel in the carotid artery blood vessel image as illustrated in FIG. 9 before diagnosing the carotid artery blood vessel image using the third artificial neural network.

The carotid ultrasound diagnostic system 200 illustrated in FIGS. 1 and 2 may further include a heat map processing unit 215 that performs heat map processing on the carotid artery blood vessel image extracted from the carotid artery extraction unit 210 and transmits the heat-map processed image to the carotid artery diagnosis unit 220, in order to increase visibility of the carotid artery blood vessel image and increase a diagnostic performance The heat map processing unit 215 may be implemented to be included in the carotid artery diagnosis unit 220. That is, the carotid artery diagnostic method according to an embodiment of the present disclosure may diagnose whether there is an abnormality in the carotid artery using a grayscale ultrasound video image of the carotid artery, and may perform the heat map processing on a grayscale ultrasound video image of the carotid artery or perform the heat map processing on the carotid artery blood vessel image extracted from the carotid ultrasound video image to diagnose whether there is an abnormality in the carotid artery.

Although not illustrated, in another modified embodiment, the carotid ultrasound diagnostic system 200 includes a carotid artery extraction unit 210 configured to extract a carotid artery blood vessel image from a carotid ultrasound video image using a pre-trained first artificial neural network, and a carotid artery diagnosis unit 220 configured to diagnose a carotid artery risk with respect to the carotid artery blood vessel image using a pre-trained second artificial neural network and output the diagnosed risk as a diagnosis result.

In the modified embodiment, the second artificial neural network corresponds to the third artificial neural network illustrated in FIG. 2.

That is, the carotid ultrasound diagnostic system 200 according to the present disclosure can construct various types of the carotid ultrasound diagnostic system 200 by combining the first artificial neural network, the second artificial neural network, and the third artificial neural network with each other. In addition, the carotid ultrasound diagnostic system 200 according to the present disclosure that can be constructed in the various types as described above further includes the heat map processing unit 215, and thus can increase visibility and a diagnostic performance and can process to expand a carotid artery blood vessel in the carotid artery blood vessel image before diagnosing whether there is an abnormality in the carotid artery. It is obvious that it is possible to extract the carotid artery and diagnose whether there is an abnormality in the carotid artery using one artificial neural network, or that it is possible to diagnose whether there is an abnormality in the carotid artery using one artificial neural network without the extraction of the carotid artery. For reference, according to experimental values, it was found that the sensitivity when a heat map image was used was entirely improved more than the sensitivity when a grayscale image was used.

A storage unit 240 illustrated in FIG. 1 stores control program data necessary for the carotid ultrasound diagnostic system 200 to control the overall operation of the medical device, and includes a database (DB) in which learning data set by the medical specialist and setting or marking information related to each learning data are stored.

The display unit 280 serves to display an interface screen for the medical specialist to set an environment, an operation mode, etc. of the carotid ultrasound diagnostic system 200, and also to display images and diagnosis results to be displayed in each operation mode (including a learning mode and a diagnostic mode). An input unit 260 displays a data input means for inputting commands, ROI settings, etc. required for a system operation by the medical specialist, etc.

Hereinafter, the carotid artery diagnostic method of the carotid ultrasound diagnostic system 200 capable of including the above-described configuration and the various combinations of the artificial neural networks is described in detail with reference to the accompanying drawings. In the following description, the present disclosure will describe diagnosing whether there is an abnormality in carotid artery by analyzing a longitudinal carotid ultrasound video image.

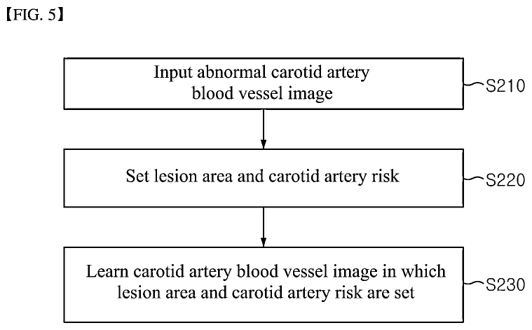

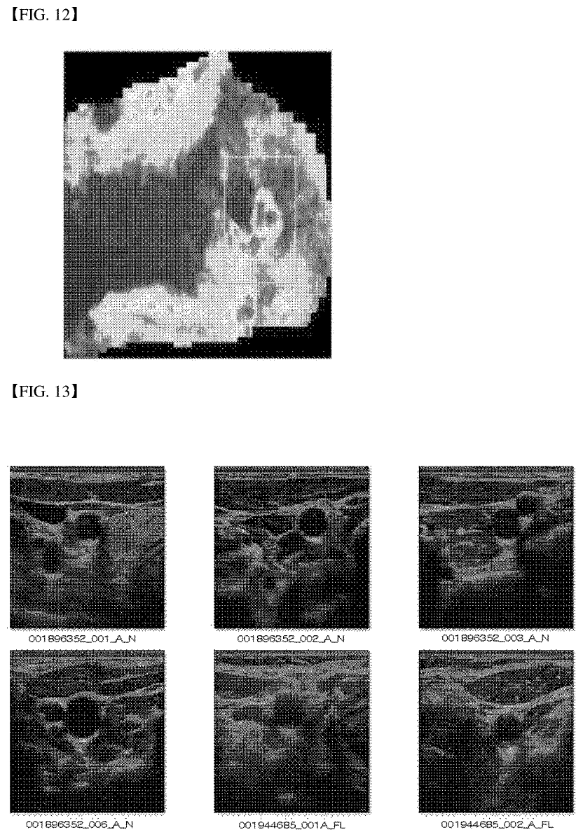

FIG. 3 illustrates a learning process of the carotid artery extraction unit 201 illustrated in FIG. 1. FIGS. 7 to 13 illustrate an operation of the carotid ultrasound diagnostic system 200 according to an embodiment of the present disclosure.

Referring to FIG. 3, first, a carotid ultrasound video image as illustrated in (a) of FIG. 7 is input to the carotid ultrasound diagnostic system 200, in step S10. The input carotid ultrasound video image is displayed on the display unit 280. The medical specialist sets an area corresponding to a carotid artery blood vessel in the displayed carotid ultrasound video image as an ROI in step S20 to execute a learning mode.

In the learning mode, the carotid artery extraction unit 210 crops the area set as the ROI by the medical specialist in the carotid ultrasound video image as illustrated in (b) of FIG. 7, in step S30. Subsequently, the carotid artery extraction unit 210 removes a noise, such as image sticking, from the cropped carotid artery blood vessel image through a filter, in step S40. Hence, when the carotid artery blood vessel image, that has been denoised, as illustrated in (c) of FIG. 7 is obtained, the carotid artery extraction unit 210 sets the denoised carotid artery blood vessel image as learning data to train a first artificial neural network, in step S50.

When the carotid artery extraction unit 210 extracts the carotid artery blood vessel image from the multiple carotid ultrasound video images through the method described above to train the first artificial neural network, the carotid artery extraction unit 210 can extract only a carotid artery blood vessel image from a carotid ultrasound video image input in a subsequent diagnostic mode.

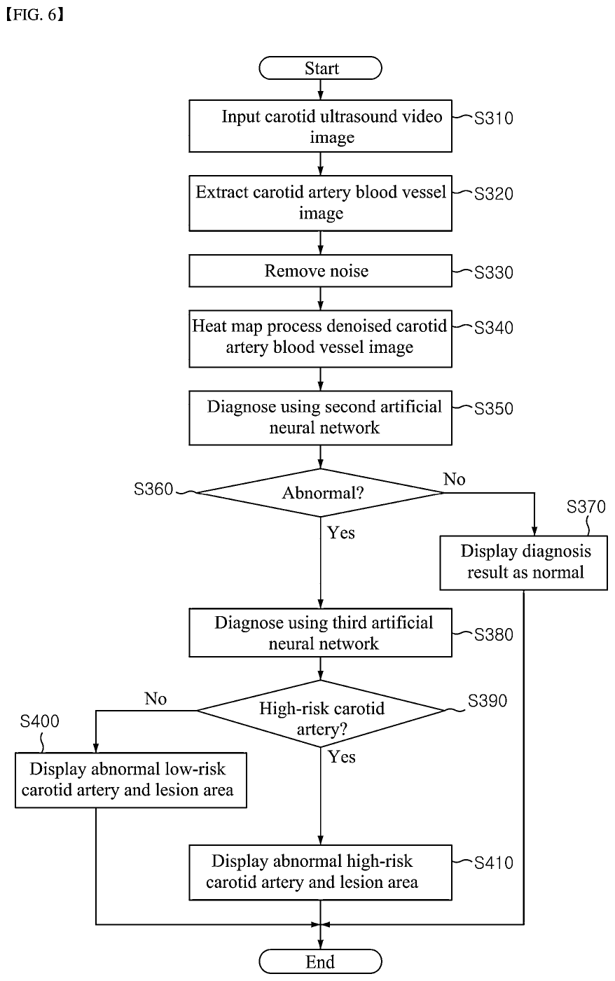

FIG. 4 illustrates a learning process of the carotid artery diagnosis unit 220 illustrated in FIG. 1.

First, the carotid artery blood vessel image that the carotid artery extraction unit 210 uses to train the first artificial neural network is input or transmitted to the carotid artery diagnosis unit 220, in step S110. If the carotid ultrasound diagnostic system 200 including the heat map processing unit 215 is provided, a grayscale image of carotid artery blood vessel obtained from the carotid artery extraction unit 210 is preferentially heat-map processed as illustrated in FIG. 8, in step S120. The heat-map processed carotid artery blood vessel image is displayed on the display unit 280.

Hence, the medical specialist reads the heat-map processed carotid artery blood vessel image and marks diagnosis information simply indicating whether the carotid artery is normal or abnormal, in step S130. The marking of the diagnosis information is to mark on a user interface screen displayed on the display unit 280. The user interface screen may include an area on which the heat-map processed carotid artery blood vessel image is displayed, a marking area of the diagnosis information, longitudinal and transverse carotid artery blood vessel setting areas, an examinee information display area, and the like. The user interface screen may be displayed and controlled by a screen display controller (not shown) constituting the carotid ultrasound diagnostic system 200, and the areas constituting the user interface screen may also be partitioned to display various information.

When the medical specialist marks the diagnosis information for the heat-map processed carotid artery blood vessel image, and then commands the learning, the carotid artery diagnosis unit 220 sets the carotid artery blood vessel image, on which the diagnosis information is marked, as learning data to train a second artificial neural network, in step S140.

When the carotid artery diagnosis unit 220 trains the second artificial neural network based on the multiple heat-map processed carotid artery blood vessel images on which the diagnosis information is marked through the method described above, the carotid artery diagnosis unit 220 can automatically diagnose whether a heat-map processed carotid artery blood vessel image input in a subsequent diagnostic mode is normal or abnormal.

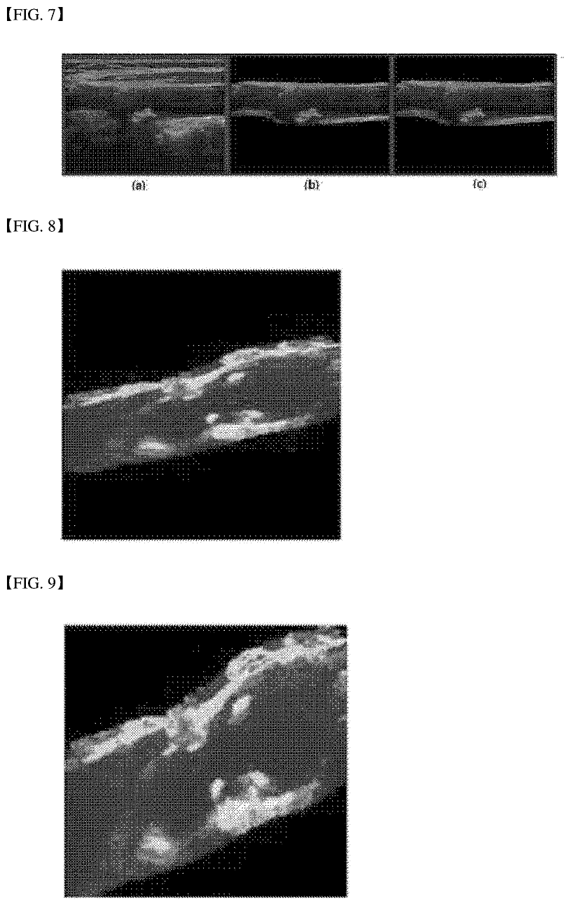

FIG. 5 illustrates a learning process of the carotid artery diagnosis unit 220 further including a third artificial neural network.

First, when an image diagnosed as abnormal among the carotid artery blood vessel images that the carotid artery diagnosis unit 220 uses to train the second artificial neural network is input in step S210, the image is also displayed on the user interface screen of the display unit 280. The displayed carotid artery blood vessel image is a heat-map processed image.

The medical specialist reads the heat-map processed abnormal carotid artery blood vessel image to set a lesion area where plaque is excessively located, a lesion area where the thrombus is excessively distributed in the blood vessel, a lesion area where the possibility of thrombus separation is high, a lesion area where the carotid artery stenosis is visible, and the like, as boxes, and sets a carotid artery risk for each lesion area set as the box, for example, an abnormal high-risk carotid artery and an abnormal low-risk carotid artery, in step S220.

Subsequently, when the medical specialist commands learning for the carotid artery blood vessel image in which the lesion area and the carotid artery risk are set, the carotid artery diagnosis unit 220 sets the carotid artery blood vessel image, in which the lesion area and the carotid artery risk are set, as learning data to train the third artificial neural network, in step S230.

When the carotid artery diagnosis unit 220 trains the third artificial neural network through the method described above, the carotid artery diagnosis unit 220 can automatically diagnose carotid artery risk information as well as the lesion area for the carotid artery blood vessel image that is first diagnosed as an abnormal carotid artery in a subsequent diagnostic mode.

A process of automatically diagnosing whether there is an abnormality in the carotid artery using the artificial neural networks that have learned the learning data through the above-described learning process is additionally described below.

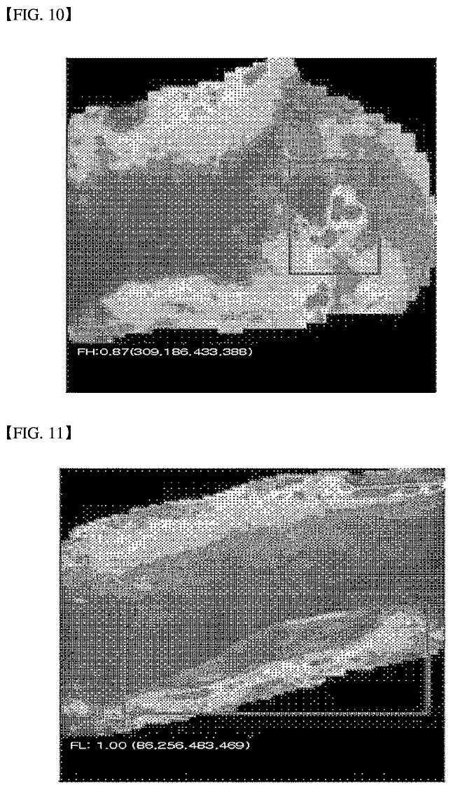

FIG. 6 illustrates a diagnostic process of the carotid ultrasound diagnostic system 200 according to an embodiment of the present disclosure.

In a diagnostic mode, first, a carotid ultrasound video image can be input through the carotid ultrasound video image acquisition unit 100, in step S310. It may be assumed that the input carotid ultrasound video image is the same as the carotid ultrasound video image illustrated in (a) of FIG. 7, and the carotid ultrasound video image is input to the carotid artery extraction unit 210.

The carotid artery extraction unit 210 extracts a carotid artery blood vessel image as illustrated in (b) of FIG. 7 from the carotid ultrasound video image using a pre-trained first artificial neural network in step S320, and denoises the extracted carotid artery blood vessel image in step S330 to generate a carotid artery blood vessel image as illustrated in (c) of FIG. 7.

Next, the denoised carotid artery blood vessel image is transmitted to the carotid artery diagnosis unit 220. If the carotid ultrasound diagnostic system 200 including the heat map processing unit 215 is provided, the carotid artery blood vessel image is heat-map processed by the heat map processing unit 215 in step S340, and the heat-map processed carotid artery blood vessel image as illustrated in FIG. 8 is transmitted to the carotid artery diagnosis unit 220.

The carotid artery diagnosis unit 220 diagnoses whether the carotid artery for the heat-map processed carotid artery blood vessel image is normal using a pre-trained second artificial neural network, in step S350. When a diagnosis result is diagnosed as normal in step S360, the diagnosis result is displayed on the display unit 280 as normal in step S370, and a series of diagnosis processes are terminated.

When the diagnosis result is diagnosed as abnormal, the diagnosis result is simply displayed as abnormal if the carotid artery diagnosis unit 220 does not include a third artificial neural network. On the other hand, if the carotid artery diagnosis unit 220 includes the third artificial neural network, the carotid artery diagnosis unit 220 processes to expand a carotid artery blood vessel in the carotid artery blood vessel image as illustrated in FIG. 9 before diagnosing the carotid artery blood vessel image using the third artificial neural network. Such an expansion of the carotid artery blood vessel is one selectable option.

Next, the carotid artery diagnosis unit 220 that processes to expand the carotid artery blood vessel diagnoses a carotid artery risk with respect to the carotid artery blood vessel image using the pre-trained third artificial neural network, in step S380. If a diagnosis result of the carotid artery risk is a high-risk carotid artery in step S390, step S410 proceeds, and the display unit 280 displays an abnormal high-risk (FH) carotid artery and a lesion area (bounding box) as illustrated in FIG. 10. If the diagnosis result of the carotid artery risk is a low-risk carotid artery, step S400 proceeds, and the display unit 280 displays an abnormal low-risk (FL) carotid artery and a lesion area (bounding box) as illustrated in FIG. 11, and then a series of diagnosis processes are terminated.

For reference, FIG. 10 illustrates a high-risk carotid artery blood vessel image diagnosed and displayed by the carotid ultrasound diagnostic system 200 according to an embodiment of the present disclosure, and FIG. 12 illustrates an image where the medical specialist has marked a lesion area (more specifically, a location of plaque). Comparing the images of FIGS. 10 and 12, it can be seen that a lesion location of the automatically diagnosed high-risk (FH) carotid artery coincides with the lesion area marked by the medical specialist. That is, this implies that the diagnostic accuracy of the carotid ultrasound diagnostic system 200 according to an embodiment of the present disclosure is high.

As described above, the carotid ultrasound diagnostic method of the carotid ultrasound diagnostic system 200 according to an embodiment of the present disclosure automatically diagnoses whether there is an abnormality in carotid artery with respect to a carotid ultrasound video image input or transmitted through the carotid ultrasound video image acquisition unit 100 or read from a memory using one or more artificial neural networks and also displays a risk and a lesion area of the carotid artery together, and thus the present disclosure can provide an effect of accurately detecting even a sign minutely displayed on the carotid ultrasound video image without being dependent on the reading ability of each examiner (reader).

Furthermore, the present disclosure can be implemented by an independent ultrasound diagnostic device and can also be implemented by a telemedicine server, and thus the present disclosure has an advantage of being able to provide telemedicine services. In addition, the present disclosure can detect and display in advance a thrombus with high separability that cannot be identified with the naked eye of an examiner, and thus the present disclosure can prevent in advance a stroke risk by taking necessary measures in advance for examinees with a possibility of thrombus floating, examinees with carotid artery stenosis, and the like.

The embodiment of the present disclosure described above describes automatically diagnosing whether there is an abnormality in the carotid artery by learning a longitudinal carotid ultrasound video image. However, the present disclosure may automatically diagnose whether there is an abnormality in the carotid artery by learning a transverse carotid ultrasound video image as illustrated in FIG. 13, and may automatically diagnose whether there is an abnormality in the carotid artery by learning both longitudinal and transverse carotid ultrasound video images. In some embodiments, if results of automatically diagnosing whether there is an abnormality in the carotid artery with respect to both longitudinal and transverse carotid ultrasound video images do not match, the present disclosure can finally diagnose whether the carotid artery for any one image diagnosed as normal or abnormal is normal or abnormal using a newly trained artificial neural network.

Furthermore, the present disclosure can reinforce learning data in a way of cropping each variable bounding box while randomly varying a size of a bounding box around a lesion area marked by the medical specialist in multiple steps, in order to reinforce the learning data.

The present disclosure can accommodate lesions of various sizes and proportions by cropping the image at various sizes without cropping the image at a fixed size as described above, and thus the present disclosure can secure high-quality learning data, and as a result, obtain an effect of improving the performance of the artificial neural networks.

Based on the description of embodiments above, those skilled in the art can clearly understand that the present disclosure can be achieved through a combination of software and hardware or can be achieved only with hardware. The objects of the technical solution of the present disclosure or parts contributing to the prior arts can be implemented in the form of program instructions that can be executed through various computer components, and can be recorded in a machine-readable recording medium. The machine-readable recording medium may include program instructions, data files, data structures, and the like alone or in combination. The program instructions recorded on the machine-readable recording medium may be specially designed and configured for the present disclosure, or may be known and available to those skilled in the art of computer software.

Examples of the program instructions include not only a machine code, such as generated by a compiler, but also a high-level language code that can be executed by a computer using an interpreter or the like. A hardware device may be configured to operate as one or more software modules to perform the processing according to the present disclosure, and vice versa. The hardware device may be coupled with a memory (storage unit) such as ROM/RAM for storing program instructions as illustrated in FIG. 1, and may include a processor such as a CPU or GPU configured to execute the program instructions stored in the memory and may include a communication unit capable of sending and receiving signals to and from an external device. In addition, the hardware device may include a keyboard, a mouse, and other external input devices for receiving commands written by developers.

So far, although the present disclosure has been described with reference to specific details such as specific components and limited embodiments and drawings, these are merely provided to help a more general understanding of the present disclosure. The present disclosure is not limited to the above embodiments, and various modifications and variations can be made from these descriptions by those of ordinary skill in the art to which the present disclosure pertains. Accordingly, the spirit of the present disclosure should not be defined to be limited to the embodiments described above, and appended claims and all modifications equally or equivalently to the appended claims shall fall within the scope of the spirit of the present disclosure.

1-12. (canceled)

13. A carotid ultrasound diagnostic method capable of being executed in a computer system capable of reading a carotid ultrasound video image, the carotid ultrasound diagnostic method comprising:
a first step of extracting a carotid artery blood vessel image from an input or received carotid ultrasound video image or a carotid ultrasound video image accessed from a storage unit using a pre-trained artificial neural network; and
a second step of diagnosing whether a carotid artery for the extracted carotid artery blood vessel image is normal using a pre-trained artificial neural network and displaying and outputting a diagnosis result.

14. The carotid ultrasound diagnostic method of claim 13, further comprising:
a third step of, when the diagnosis result is abnormal, further diagnosing a carotid artery risk with respect to the extracted carotid artery blood vessel image using a pre-trained artificial neural network and displaying and outputting the diagnosed carotid artery risk as the diagnosis result.

15. The carotid ultrasound diagnostic method of claim 14, wherein in the third step, a lesion area in the extracted carotid artery blood vessel image is displayed and output together.

16. The carotid ultrasound diagnostic method of claim 14, further comprising:
immediately before the third step, processing to expand a carotid artery blood vessel in the extracted carotid artery blood vessel image.

17. The carotid ultrasound diagnostic method of claim 13, further comprising:
before the second step, performing a heat-map processing on the extracted carotid artery blood vessel image.

18. The carotid ultrasound diagnostic method of claim 14, further comprising:
before the second step, performing a heat-map processing on the extracted carotid artery blood vessel image.

19. The carotid ultrasound diagnostic method of claim 15, further comprising:
before the second step, performing a heat-map processing on the extracted carotid artery blood vessel image.

20. The carotid ultrasound diagnostic method of claim 16, further comprising:
before the second step, performing a heat-map processing on the extracted carotid artery blood vessel image.

21. The carotid ultrasound diagnostic method of claim 17, wherein the first step uses a first artificial neural network that is trained by cropping an area set as a region of interest (ROI) by a medical specialist in one or more carotid ultrasound video images and setting a carotid artery blood vessel image that is denoised as learning data.

22. The carotid ultrasound diagnostic method of claim 18, wherein the first step uses a first artificial neural network that is trained by cropping an area set as a region of interest (ROI) by a medical specialist in one or more carotid ultrasound video images and setting a carotid artery blood vessel image that is denoised as learning data.

23. The carotid ultrasound diagnostic method of claim 19, wherein the first step uses a first artificial neural network that is trained by cropping an area set as a region of interest (ROI) by a medical specialist in one or more carotid ultrasound video images and setting a carotid artery blood vessel image that is denoised as learning data.

24. The carotid ultrasound diagnostic method of claim 20, wherein the first step uses a first artificial neural network that is trained by cropping an area set as a region of interest (ROI) by a medical specialist in one or more carotid ultrasound video images and setting a carotid artery blood vessel image that is denoised as learning data.

25. The carotid ultrasound diagnostic method of claim 17, wherein the second step uses a second artificial neural network that is trained by setting one or more carotid artery blood vessel images, that are marked as normal or abnormal by a medical specialist, as learning data.

26. The carotid ultrasound diagnostic method of claim 18, wherein the second step uses a second artificial neural network that is trained by setting one or more carotid artery blood vessel images, that are marked as normal or abnormal by a medical specialist, as learning data.

27. The carotid ultrasound diagnostic method of claim 19, wherein the second step uses a second artificial neural network that is trained by setting one or more carotid artery blood vessel images, that are marked as normal or abnormal by a medical specialist, as learning data.

28. The carotid ultrasound diagnostic method of claim 20, wherein the second step uses a second artificial neural network that is trained by setting one or more carotid artery blood vessel images, that are marked as normal or abnormal by a medical specialist, as learning data.

29. The carotid ultrasound diagnostic method of claim 14, wherein the third step uses a third artificial neural network that is trained by setting a lesion area set by a medical specialist in one or more carotid artery blood vessel images and a carotid artery risk set for the lesion area, as learning data.

30. The carotid ultrasound diagnostic method of claim 15, wherein the third step uses a third artificial neural network that is trained by setting a lesion area set by a medical specialist in one or more carotid artery blood vessel images and a carotid artery risk set for the lesion area, as learning data.

31. The carotid ultrasound diagnostic method of claim 16, wherein the third step uses a third artificial neural network that is trained by setting a lesion area set by a medical specialist in one or more carotid artery blood vessel images and a carotid artery risk set for the lesion area, as learning data.

32. A carotid ultrasound diagnostic method capable of being executed in a computer system capable of reading a carotid ultrasound video image, the carotid ultrasound diagnostic method comprising:
diagnosing whether a carotid artery is normal in an input or received carotid ultrasound video image or a carotid ultrasound video image accessed from a storage unit using a pre-trained artificial neural network;
when a diagnosis result is abnormal, further diagnosing a carotid artery risk using the artificial neural network; and
displaying and outputting one of diagnosing whether the carotid artery is normal and diagnosing the carotid artery risk as the diagnosis result.

33. The carotid ultrasound diagnostic method of claim 32, wherein a lesion area is displayed and output together with the carotid artery risk.

34. The carotid ultrasound diagnostic method of claim 32, wherein when the diagnosis result is abnormal, the carotid artery risk is diagnosed by processing to expand a carotid artery blood vessel capable of being extracted from the carotid ultrasound video image.

35. The carotid ultrasound diagnostic method of claim 32, further comprising:
before diagnosing whether the carotid artery is normal, performing a heat-map processing on the carotid ultrasound video image.

36. The carotid ultrasound diagnostic method of claim 33, further comprising:
before diagnosing whether the carotid artery is normal, performing a heat-map processing on the carotid ultrasound video image.

37. The carotid ultrasound diagnostic method of claim 34, further comprising:
before diagnosing whether the carotid artery is normal, performing a heat-map processing on the carotid ultrasound video image.
